(19) United States
(12) Patent Application Publication
(10) Pub. No.: US 2026/0087187 A1
(43) Pub. Date: Mar. 26, 2026

(54) APPARATUS AND METHOD FOR OPTIMIZING PROPERTY VALUES OF THREE-DIMENSIONAL OBJECT BY VOLUME OF HOMOGENEOUS PROPERTIES

(71) Applicant: FIELDCURE CO., LTD., Seoul (KR)

(72) Inventors: Jong Hyun KIM, Seoul (KR); Sang Min PARK, Seoul (KR)

(73) Assignee: FIELDCURE CO., LTD., Seoul (KR)

(21) Appl. No.: 19/111,814

(22) PCT Filed: Sep. 14, 2023
(86) PCT No.: PCT/KR2023/013790

§ 371 Date: Mar. 14, 2025

(30) Foreign Application Priority Data
Sep. 16, 2022 (KR) 10-2022-0116946
Sep. 8, 2023 (KR) 10-2023-0119398

(51) Int. Cl. G06F 30/12; A61B 5/0536; G06T 7/11; G06T 7/13; G06T 17/00

(52) U.S. Cl. CPC G06F 30/12; A61B 5/0536; G06T 7/11; G06T 7/13; G06T 17/00; G06T 2200/04; G06T 2207/10072; G06T 2207/30004; G06T 2210/41

(57) ABSTRACT

According to an embodiment of the present invention, an apparatus for optimizing property values of a three-dimensional object by volume of homogeneous properties is an apparatus for optimizing property values of a three-dimensional object, which are unique properties of a material, the apparatus comprising: a homogenous property volume division unit that divides a three-dimensional object into volumes of homogenous properties by which properties are distinguished; an initial property value allocation unit that allocates initial property values to the divided volumes, respectively; an electrode arrangement unit that arranges a plurality of electrodes on the surface of the three-dimensional object; a measurement condition setting unit that sets at least one of a voltage and current applied to the plurality of arranged electrodes; a measurement result derivation unit that applies at least one of the voltage and current to the plurality of electrodes according to the setting of the measurement condition setting unit and measures at least one of a voltage and current generated in the plurality of electrodes; and a volume-specific property value optimization unit that performs a simulation according to the same conditions as the electrode arrangement unit and the measurement condition setting unit, and determines property values by volume to minimize a difference between a measurement value from the measurement result derivation unit and a simulation performance value. Therefore, there is an effect of dividing the three-dimensional object into volumes of homogeneous properties and then deriving optimized property values by volume of homogeneous properties.

### CROSS-REFERENCE TO RELATED APPLICATION

This application is an International Application No. PCT/KR2023/013790 filed on Sep. 14, 2023, which is based on and claims the benefit of a Korean Patent Application No. 10-2023-0119398 filed on Sep. 8, 2023 in the Korean Intellectual Property Office, and of a Korean Provisional Patent Application No. 10-2022-0116946 filed on Sep. 16, 2022 in the Korean Intellectual Property Office, the disclosure of each of which is herein incorporated by reference in its entirety.

### TECHNICAL FIELD

The present invention relates to an apparatus and method for identifying and optimizing physical property values for each homogeneous property volume (i.e., a volume with identical physical properties) in a three-dimensional (3D) subject. Specifically, it provides an apparatus and method that first segment the 3D subject into homogeneous property volumes and then determine the optimal physical property values for each volume.

### BACKGROUND ART

In general, bioelectrical impedance analysis is a method of passing a small electric current through electrodes attached to the body and measuring the voltage to measure body composition using the value of the change in electrical signals by cell membranes and tissues, i.e., bioimpedance value, and is mainly used to inspect the health status of the body such as muscles, fat, cells, body mass, basal metabolism, water in the body, metabolic activity, bone minerals and plasma.

The electrical impedance tomography(EIT) is used to image physiological functions such as respiration and blood flow by attaching a plurality of electrodes to the body and repeatedly applying electric current and measuring voltage to restore the impedance distribution of the body cross-section.

These EIT inspections involve the subject wearing a vest or belt-shaped device equipped with a number of EIT electrodes. Accordingly, in recent years, various studies have been steadily conducted to improve the convenience and reliability of electrical impedance recording of subjects using EIT electrodes.

However, due to the complex structure of the subject, it remains very difficult to measure the distribution of three-dimensional physical properties, such as electrical conductivity, with sufficient resolution despite various academic efforts, and it is necessary to simplify the subject by a realistic approach. Therefore, to address this issue, an approximation method is required to identify and optimize 3D physical property values based on homogeneous property volumes (i.e., volumes with identical physical properties, such as the same type of tissue in the body).

### DISCLOSURE OF INVENTION

### Technical Problem

To address this issue, the present disclosure provides an apparatus and method for optimizing physical property values for each homogeneous property volume (i.e., a volume with identical physical properties) in a 3D subject. The proposed approach involves segmenting the 3D subject into homogeneous property volumes and subsequently determining the optimized property values for each volume.

### Solution to Problem

In order to accomplish the aforementioned object, an apparatus for optimizing physical property values for each homogeneous property volume in a 3D subject according to an embodiment of the inventive concept, wherein the property values represent intrinsic properties of substances constituting the subject, may include a homogeneous property volume division unit dividing the 3D subject into volumes based on distinct homogeneous properties; an initial property value allocation unit assigning an initial property value for each of the divided volumes; an electrode arrangement unit placing a plurality of electrodes on a surface of the 3D subject; a measurement condition setting unit setting at least one of voltage and current applied to the plurality of electrodes; a measurement result derivation unit applying at least one of voltage and current to the plurality of electrodes in accordance with the setting of the measurement condition setting unit and measuring at least one of voltage and current generated on the plurality of electrodes; and a volume property value optimization unit performing a simulation under the same conditions as the electrode arrangement unit and the measurement condition setting unit, and determining the property value for each volume to minimize the sum of squared differences between the measured values of the measurement result derivation unit and the simulated values.

Here, the volume property value optimization unit may include a volume selection module selecting at least one volume to optimize the property value among the plurality of the volumes; and a volume property value determination module optimizing a property value of the selected volume to minimize the sum of squared differences between the measured values of the measurement result derivation unit and the simulated values to determine the property value of the selected volume.

Here, the homogeneous property volume division unit may receive a medical image of the 3D subject, extract the outline of the subject based on the received medical image, and divide the 3D subject into volumes based on the distinct homogeneous properties.

Here, the homogeneous property volume division unit may receive a medical image information divided by volumes based on the homogeneous properties.

Here, when voltage is applied to the plurality of electrodes based on the setting in the measurement result derivation unit, current flowing to the plurality of electrodes may be measured.

Here, when current is applied to the plurality of electrodes based on the setting in the measurement result derivation unit, voltage between the plurality of electrodes may be measured.

Here, the initial property value assigned for each of the divided volumes in the initial property value allocation unit may be either the electrical property value measured by the conductivity measurement method or the electrical property value known in advance for the tissue of the human body.

Here, the dividing of the 3D subject into volumes based on the distinct homogeneous properties in the homogeneous property volume division unit may divide into anatomical volumes using at least one of the magnetic resonance imaging (MRI) or the computed tomography (CT).

Here, the anatomic volume may include at least one of skin, skull, white matter, gray matter of the mammalian brain, and multiple internal organs, bones, muscles and fat of the mammalian chest and abdomen.

Here, the property value of each sub-volume may be at least one of electrical conductivity and permittivity.

Here, the minimizing of the sum of squared differences between the measured values of the measurement result derivation unit and the simulated values in the volume property value optimization unit may be performed by running the simulation repeatedly while changing the property value assigned to the selected volume by using the nonlinear least-squares method to determine the property value of the selected volume.

Here, the apparatus may further include sub-volume property value optimization unit dividing the volume into a plurality of sub-volumes by using the property value of each volume determined in the volume property value optimization unit, and optimizing the property value for each sub-volume.

Here, the sub-volume property value optimization unit may include a sub-volume selection module selecting at least one sub-volume to optimize the property value from respective volumes of which property value is determined; and a sub-volume property value determination module optimizing the property value of the selected sub-volume to minimize the sum of squared differences between the measured values of the measurement result derivation unit and the simulated values to determine the property value of the selected sub-volume.

Here, the sub-volume in the sub-volume property value optimization unit may be at least one voxel forming a cube.

Here, the property value of each sub-volume may be at least one of electrical conductivity and permittivity.

Here, the minimizing of the sum of squared differences between the measured values of the measurement result derivation unit and the simulated values in the sub-volume property value optimization unit may be performed by running the simulation repeatedly while changing the property value assigned to the selected sub-volume by using the nonlinear least-squares method to determine the property value of the selected sub-volume.

In order to accomplish the aforementioned object, a method for optimizing physical property values for each homogeneous property volume in a 3D subject according to an embodiment of the inventive concept, wherein the property values represent intrinsic properties of substances constituting the subject, may include a step of homogeneous property volume division dividing the 3D subject into volumes based on distinct homogeneous properties; a step of initial property value allocation assigning an initial property value of each of the divided volumes; a step of electrode arrangement placing a plurality of electrodes on the surface of the 3D subject; a step of measurement condition set setting at least one of the voltage and current applied to the plurality of electrodes; a step of measurement result derivation applying at least one of voltage and current to the plurality of electrodes in accordance with the setting in the step of measurement condition set and measuring at least one of voltage and current generated on the plurality of electrodes; and a step of volume property value optimization performing a simulation based on the same conditions as the step of electrode arrangement and the step of measurement condition set, and determining the property value for each volume to minimize the sum of squared differences between the measured values in the step of measurement result derivation and the simulated values.

Here, the step of volume property value optimization may include a volume selection process selecting at least one volume to optimize the property value among the plurality of the volumes; and a volume property value determination process optimizing a property value of the selected volume to minimize the sum of squared differences between the measured values in the step of measurement result derivation and the simulated values to determine the property value of the selected volume.

Here, the method may further include a step of sub-volume property value optimization dividing the volume into a plurality of sub-volumes by using the property value of each volume determined in the step of volume property value optimization, and optimizing a property value for each sub-volume.

Here, the step of sub-volume property value optimization may include a sub-volume selection process selecting at least one sub-volume to optimize the property value from respective volumes of which property value is determined; and a sub-volume property value determination process optimizing a property value of the selected sub-volume to minimize the sum of squared differences between the measured values in the step of measurement result derivation and the simulated values to determine the property value of the selected volume.

### Advantageous Effects of Invention

According to the apparatus and the method for optimizing physical property values for each homogeneous property volume in a three-dimensional subject according to an embodiment of the inventive concept can derive the optimized property value for each homogeneous property volume after dividing the three-dimensional object into homogeneous property volumes

According to the inventive concept, it is possible to create a conductivity map for each type of tissue of the subject by dividing the tissue and deriving the electrical property information about the subject based on the divided tissue. In addition, more sophisticated electrical property information can be derived since data based on actual measurements are obtained.

The conductivity map generated as above can be used for various electrical stimulation therapies, and can improve the accuracy of the treatment during the brain stimulation therapy through the conductivity map, and can affect to the study and analysis of the human body.

According to the apparatus and method for optimizing property values for each homogeneous property volume in 3D subject according to an embodiment of the inventive concept, in inputting and optimizing the electrical property information for each type of tissue, it can be shortened time than the conventional technology, it can be configured relatively simply, and the value of the electric current applied to the human body can be minimized.

### BRIEF DESCRIPTION OF DRAWINGS

FIG. 1 is a view illustrating an apparatus for optimizing the physical property values for each homogeneous property volume in a 3D subject according to an embodiment of the inventive concept.

FIG. 2 is a view illustrating a method for optimizing physical property values for each homogeneous property volume in a 3D subject according to another embodiment of the inventive concept.

FIG. 3 is a view illustrating a step of volume property value optimization in a method for optimizing physical property values for each homogeneous property volume in a 3D subject according to an embodiment of the inventive concept.

FIG. 4 is a view illustrating a step of sub-volume property value optimization in a method for optimizing physical property values for each homogeneous property volume in a 3D subject according to an embodiment of the inventive concept.

FIGS. 5A to 5C are a view illustrating a method of dividing a volume into a plurality of sub-volumes in a method for optimizing physical property values for each homogeneous property volume in a 3D subject according to an embodiment of the inventive concept, and shows a cross-sectional view of the volume.

FIG. 6 is a schematic view illustrating an apparatus for optimizing the physical property values for each homogeneous property volume in a 3D subject according to another embodiment of the inventive concept.

FIG. 7 is a flow chart of a method for optimizing physical property values for each homogeneous property volume in a 3D subject according to another embodiment of the inventive concept.

FIGS. 8A and 8B show a detailed view of the model divided by tissue types in an apparatus for optimizing the physical property values for each homogeneous property volume in a 3D subject according to another embodiment of the inventive concept.

FIGS. 9A to 9C are a view of an apparatus for optimizing the physical property values for each homogeneous property volume in a 3D subject according to another embodiment of the inventive concept, (A) shows the name of each side (A1˜A4), and (B) shows the name of the individual electrode of each surface, and (C) shows an electrode to which an electric current is applied to an object and an electrode to measure the voltage at that time.

FIGS. 10A and 10B show a data measurement and reconstruction figure according to the electrical properties through a 3D model in an apparatus for optimizing the physical property values for each homogeneous property volume in a 3D subject according to another embodiment of the inventive concept.

FIG. 11 is a view illustrating a method for optimizing physical property values for each homogeneous property volume in a 3D subject according to further another embodiment of the inventive concept.

### MODE FOR THE INVENTION

Embodiments of the inventive concept will be described more fully hereinafter with reference to the accompanying drawings and let those skilled in the art implement easily. It should be noted, however, that the inventive concept is not limited to the following embodiments, and may be implemented in various forms.

For clarity, shapes not related to description are excluded and the same reference designators denote the same or similar elements throughout the specification.

If it is stated that a part is connected to another part, it includes a case of ‘connecting directly’ with each other by interposing another device as well as a case of ‘connecting electrically’ with each other. Additionally, if it is stated that any part ‘include(s)’ a component, it means that other components are not excluded but further includes another component unless a specific contrary statement is described.

If there is referred that a part ‘on’ another part, it is on the part or another part may be interposed therebetween. In contrast, if there is referred that a part ‘directly on’ another part, any part is not interposed therebetween.

Terms of a first, a second, a third will be used to describe various parts, elements, regions, layers and/or sections, but not limited to these. These terms are used only to distinguish a part, element, region, layer or section with another part, element, region, layer or section. Therefore, a first part, element, region, layer or section may be described hereinafter a second part, element, region, layer or section within a scope of the inventive concept.

The professional terms will be used hereinafter in order to mention only specific embodiment, but it does not intend to limit the inventive concept. The wording in the singular used here will include the plural unless obvious opposite meaning is expressed. The meaning of ‘include’ used in this description will embody curtain feature, region, integer number, step, action, component and/or element, but not exclude exist or supplement of other feature, region, integer, step, action, component and/or element.

Terms of opposite spaces such as ‘up’ and ‘down’ may be used in order to describe easily a relationship a part with another part illustrated in a drawing. These terms will be intended to include another meaning or operation of utilized device with the meaning intended in drawings. For example, when the device on the drawing is turned over, a part described as being ‘under’ other parts are described as being ‘on’ the other parts. So, illustrative term ‘under’ includes both Referring of up and down. The device may be rotated 90 degrees or other angles, and terms denoting relative space are interpreted accordingly.

All terms including technical terms and scientific terms used herein, even if not defined differently, have the same meaning as commonly understood by those skilled in the art of the inventive concept. Terms defined in commonly used dictionaries are additionally interpreted as having meanings correspondent with related technical documents and current described content, and not interpreted in ideal or very formal meanings unless defined.

An embodiment will now be described fully hereinafter with reference to the accompanying drawings in order to implement facilely the invention inventive concept by those skilled in the art.

However, the inventive concept may be implemented in various forms and not limited to the following some embodiments.

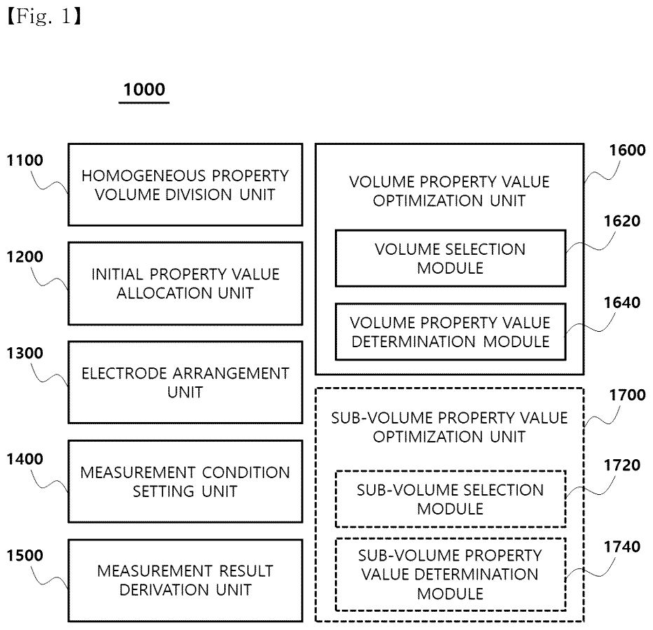

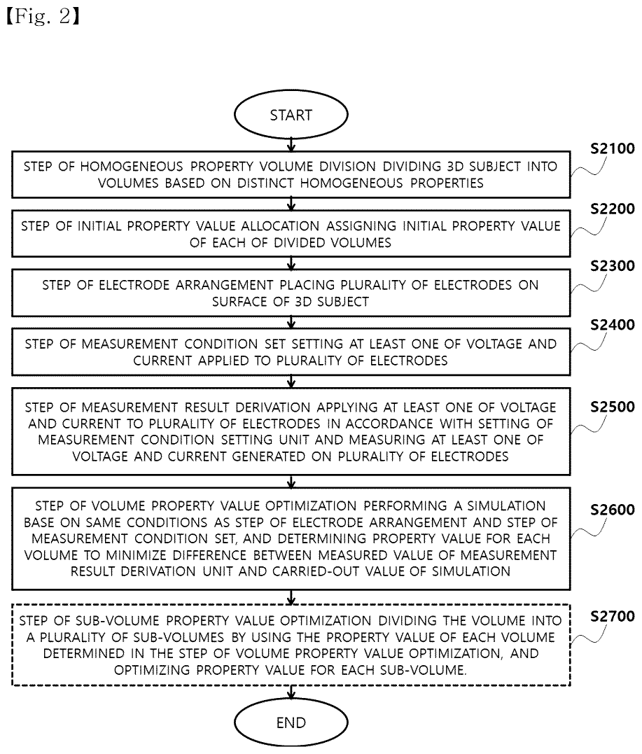

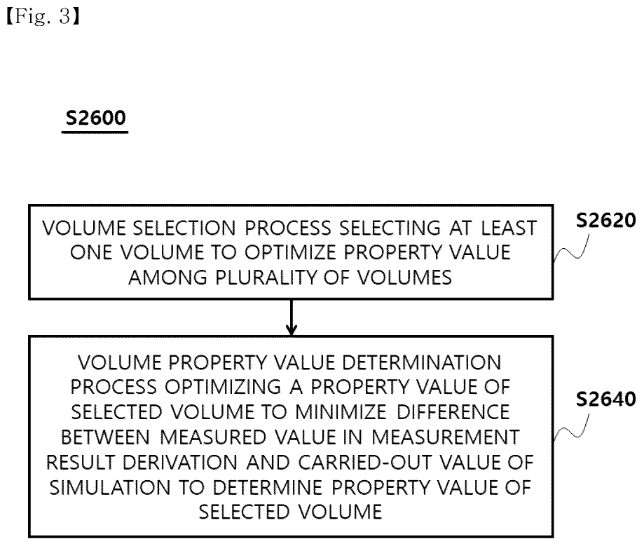

FIG. 1 is a view illustrating an apparatus for optimizing the physical property values for each homogeneous property volume in a 3D subject according to an embodiment of the inventive concept. FIG. 2 is a view illustrating a method for optimizing physical property values for each homogeneous property volume in a primary subject according to another embodiment of the inventive concept. FIG. 3 is a view illustrating a step of volume property value optimization in a method for optimizing physical property values for each homogeneous property volume in a 3D subject according to an embodiment of the inventive concept.

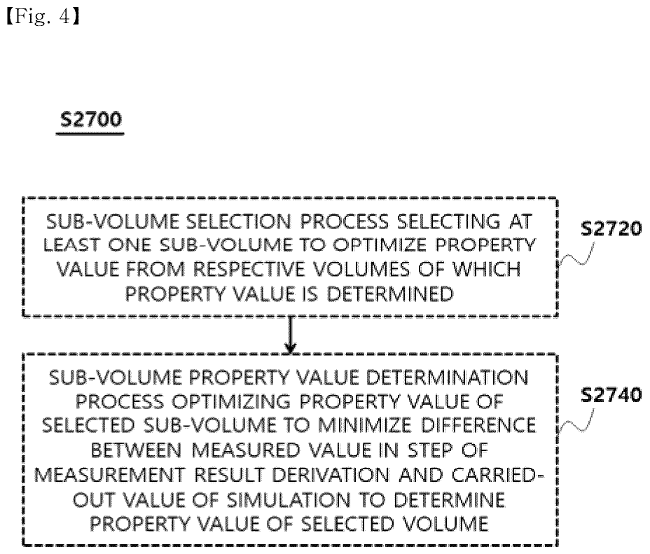

FIG. 4 is a view illustrating a step of sub-volume property value optimization in a method for optimizing physical property values for each homogeneous property volume in a 3D subject according to an embodiment of the inventive concept.

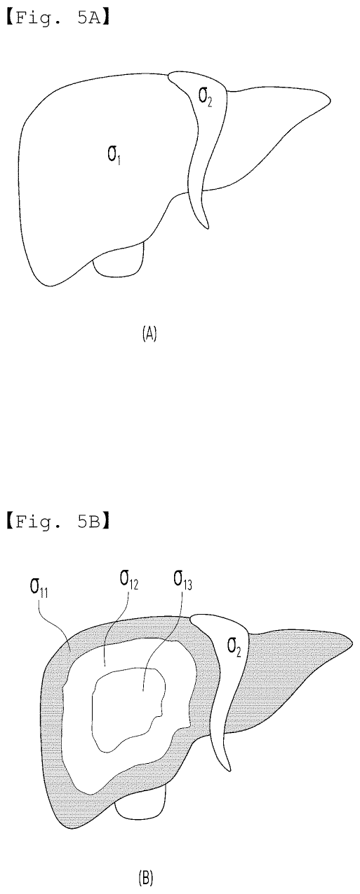

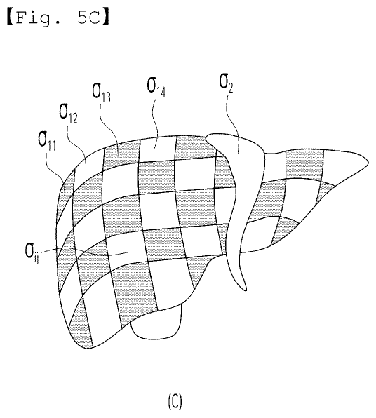

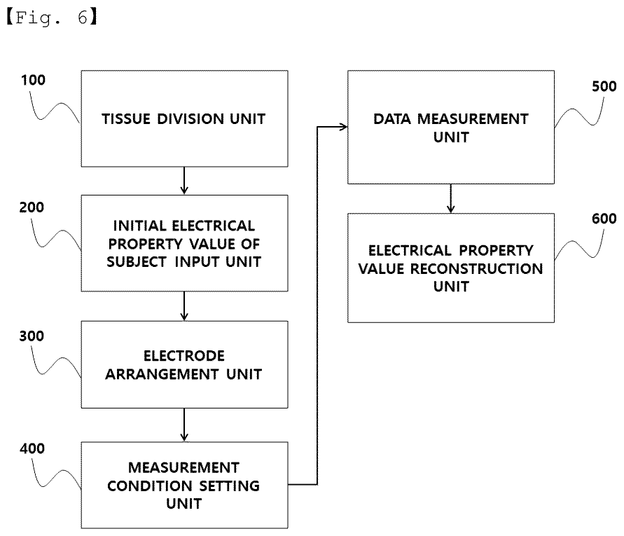

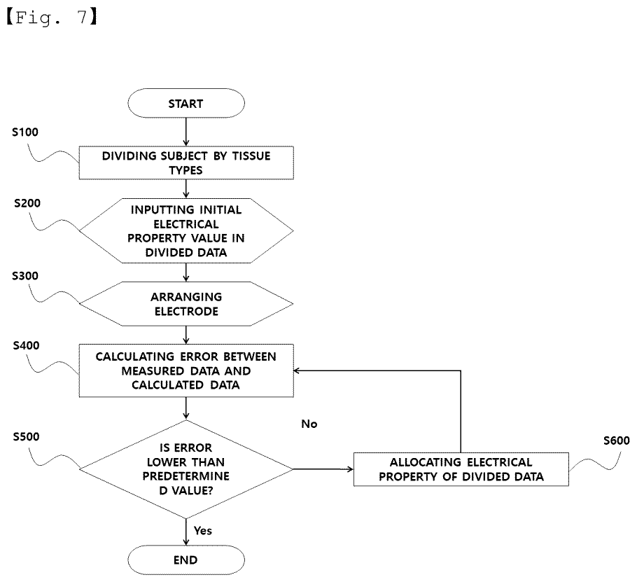

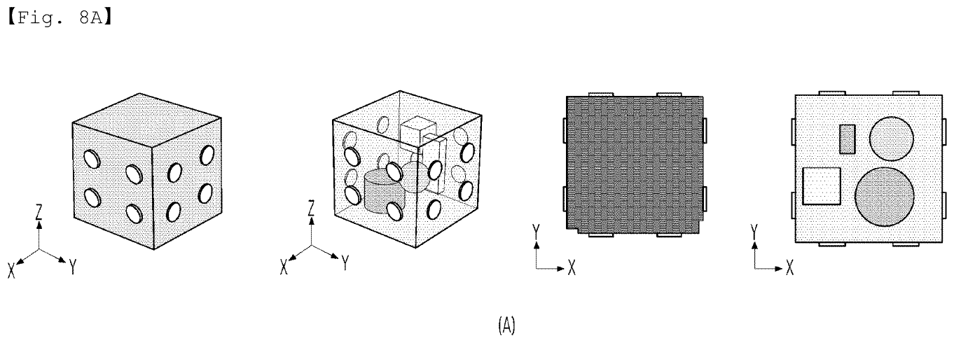

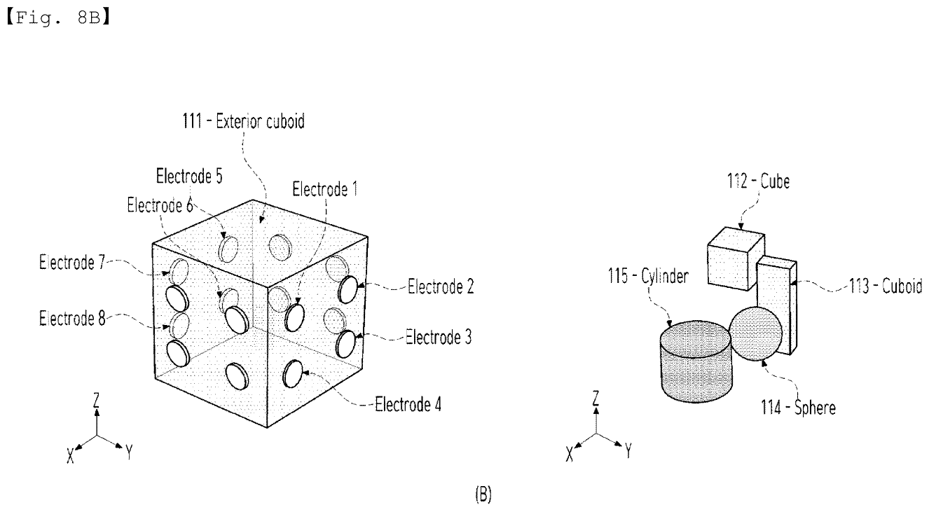

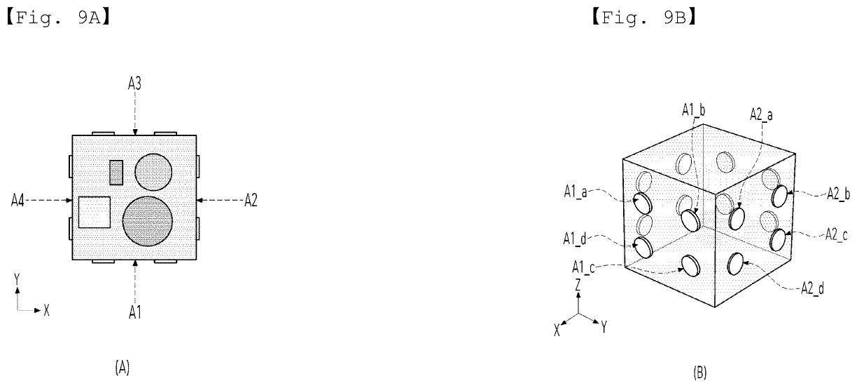

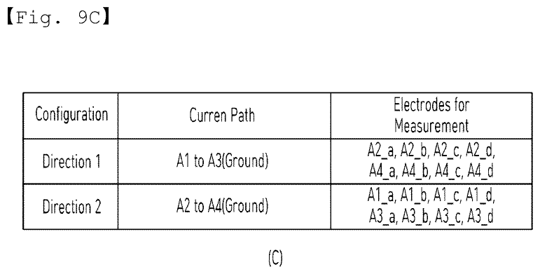

FIGS. 5A to 5C are a schematic view illustrating an apparatus for optimizing physical property values for each homogeneous property volume in a 3D subject according to another embodiment of the inventive concept. FIG. 6 is a flow chart of a method for optimizing physical property values for each homogeneous property volume in a 3D subject according to another embodiment of the inventive concept. FIG. 7 shows a view of the model divided by tissue types in an apparatus for optimizing physical property values for each homogeneous property volume in a 3D subject according to another embodiment of the inventive concept. FIG. 8 shows a detailed view of the model divided by tissue types in an apparatus for optimizing physical property values for each homogeneous property volume in a 3D subject according to another embodiment of the inventive concept. FIG. 9 is a view of an apparatus for optimizing physical property values for each homogeneous property volume in a 3D subject according to another embodiment of the inventive concept, (A) shows the name of each side (A1˜A4), and (B) shows the name of the individual electrode of each surface, and (C) shows an electrode to which an electric current is applied to an object and an electrode to measure the voltage at that time.

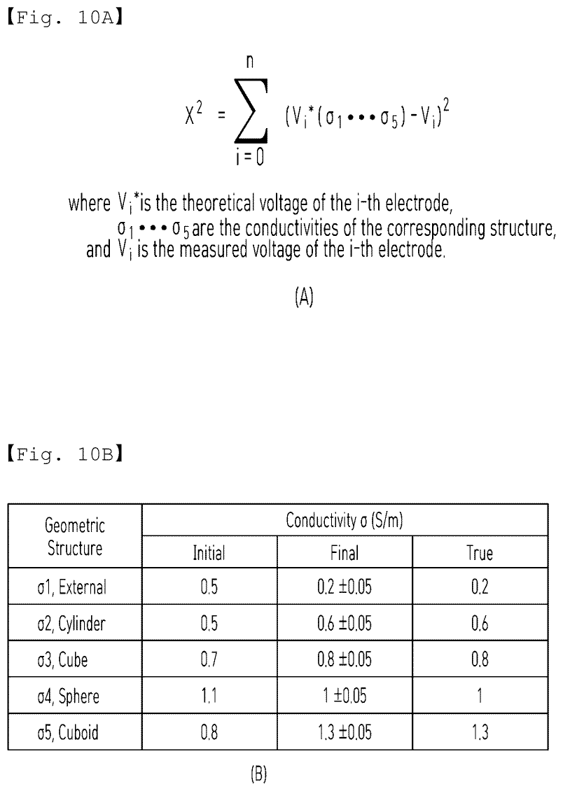

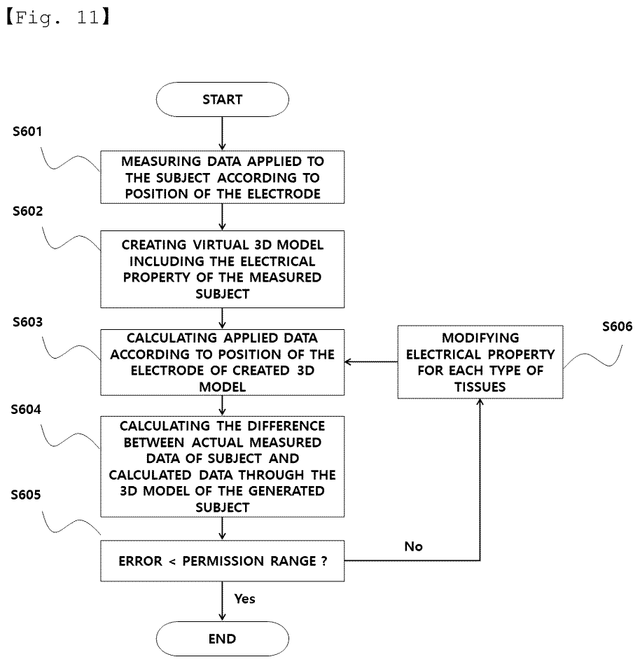

FIG. 10 shows a data measurement and reconstruction figure according to the electrical properties through a 3D model in an apparatus for optimizing property values for each homogeneous property volume in a 3D subject according to another embodiment of the inventive concept. FIG. 11 is a view illustrating a method for optimizing physical property values for each homogeneous property volume in a 3D subject according to further another embodiment of the inventive concept.

FIG. 12 shows an optimized subject of electrical properties in an apparatus for optimizing physical property values for each homogeneous property volume in a 3D subject according to another embodiment of the inventive concept.

Referring to FIG. 1, an apparatus for optimizing physical property values for each homogeneous property volume in a 3D subject according to an embodiment of the inventive concept in order to accomplish the aforementioned object is an apparatus 1000, wherein the property values represent intrinsic properties of substances constituting the subject. The apparatus may include a homogeneous property volume division unit 1100 dividing the 3D subject into volumes based on distinct homogeneous properties; an initial property value allocation unit 1200 assigning an initial property value for each of the divided volumes; an electrode arrangement unit 1300 placing a plurality of electrodes on the surface of the 3D subject; a measurement condition setting unit 1400 setting at least one of voltage and current applied to the plurality of electrodes; a measurement result derivation unit 1500 applying at least one of voltage and current to the plurality of electrodes in accordance with the setting of the measurement condition setting unit 1400 and measuring at least one of voltage and current generated on the plurality of electrodes; and a volume property value optimization unit 1600 performing a simulation based on the same conditions as the electrode arrangement unit 1300 and the measurement condition setting unit 1400 and determining the property value for each volume to minimize the sum of squared differences between the measured values of the measurement result derivation unit 1500 and the simulated values.

The homogeneous property volume division unit 1100 may divide the 3D subject into volumes based on the distinct homogeneous properties. In other words, it may arrange a part with a homogeneous property into one volume, and divide each volume based on the distinct homogeneous properties.

In particular, the homogeneous property volume division unit 1100 may receive a medical image of the 3D subject, extract the outline of the subject based on the received medical image, and divide the 3D subject into volumes based on the distinct homogeneous properties. In other words, after analyzing the medical image of the 3D subject, a part with homogeneous property may be divided into one volume in order to distinguish properties.

In addition, the homogeneous property volume division unit 1100 may receive a medical image information divided by volumes based on the homogeneous properties. This may mean that the medical image of the 3D subject may be the medical image information divided by each homogeneous property volume whose property is already distinguished.

In particular, the property value of each volume may be at least one of electrical conductivity and permittivity, and consequently, the division by the homogeneous property volume may be to divide the part with homogeneous electrical conductivity into one volume, or to divide the part with homogeneous permittivity into one volume.

The initial property value allocation unit 1200 may assign the initial property for each of the divided volumes. The initial property value assigned for each of the divided volumes in the initial property value allocation unit 1200 may either the electrical property value measured by the conductivity measurement method or the electrical property value known about the tissue of the human body, in advance.

In other words, the initial property value allocation unit 1200 may assign the electrical property value measured by the conductivity measurement method or the electrical property value known about the tissue of the human body, in advance, for each of the divided volumes.

The electrode arrangement unit 1300 may place a plurality of electrodes on the surface of the 3D subject. This may place a plurality of the electrodes in the most efficient position in order to repeat the injection of current and the measurement of voltage after attaching a plurality of the electrodes according to the electrical impedance imaging(EIT).

The measurement condition setting unit 1400 may set at least one of voltage and current applied to the plurality of the electrode. As above, at least one of voltage and current may be set in order to perform the most efficient measurement through the EIT. The method of applying a electric signal may be four ways of a constant voltage, a constant current, a constant resistance and a constant power.

Here, the frequency is constant, so the constant resistance and the constant power may be basically the same as the constant current.

The measurement result derivation unit 1500 may apply at least one of voltage and current to the plurality of electrodes according to the setting of the measurement condition setting unit 1400, and measure at least one of the voltage and the current generated on the plurality of electrodes.

In other words, the current flowing to the plurality of electrode may be measured in the case of applying voltage to the plurality of electrodes according to the setting in the measurement result derivation unit 1500, or the voltage between the plurality of electrode may be measured in the case of applying current to the plurality of electrodes.

After all, current or voltage may be measured depending on the case of applying voltage or current to the plurality of electrodes, respectively.

On the other hand, the division of the 3D subject into the distinct homogeneous property volumes in the homogeneous property volume division unit 1100 may divide into the anatomical volumes using at least one of the magnetic resonance imaging (MRI) and computed tomography (CT).

In particular, the anatomic volume may include at least one of skin, skull, white matter, gray matter of the mammalian brain, and multiple internal organs, bones, muscles and fat of the mammalian chest and abdomen.

The volume property value optimization unit 1600 may perform a simulation under the same conditions as the electrode arrangement unit 1300 and the measurement condition setting unit 1400, and the measurement result derivation unit 1500 may determine the property value for each volume to minimize the sum of squared differences between the measured values and the simulated values.

For example, according to the setting of the electrode arrangement unit 1300, the measurement condition setting unit 1400 and the measurement result derivation unit 1500, the measurement result derivation unit 1500 may derive the measured values and the simulated values may be derived after performing a simulation by the same setting as the condition of the electrode arrangement unit 1300 and the measurement condition setting unit 1400 such that the property value for each volume is determined to minimize the sum of squared differences between the measured values and the simulated values.

The volume property value optimization unit 1600 may include a volume selection module 1620 selecting at least one volume to optimize the property value among the plurality of the volumes; and a volume property value determination module 1640 optimizing a property value of the selected volume to minimize the sum of squared differences between the measured values in the measurement result derivation and the simulated values to determine the property value of the selected volume.

In other words, the volume selection module 1620 may select at least one volume to optimize physical property values among the plurality of volumes, and repeatedly perform the process for optimizing the physical property values of the selected volume to minimize the sum of squared differences between the measured values of the measurement result derivation unit 1500 and the simulated values, so that the volume property value determination module 1640 can determine the physical property values of the selected volume.

In particular, the minimizing of the sum of squared differences between the measured values of the measurement result derivation unit 1500 and the simulated values in the volume property value optimization unit 1600 may be performed by running the simulation repeatedly while changing the property value assigned to the selected volume to determine the property value of the selected volume by using the nonlinear least-squares method.

Here, the inventive concept may further include a sub-volume property value optimization unit 1700 dividing the volume into a plurality of sub-volumes by using the property value of each volume determined by the volume property value optimization unit 1600 and optimizing a property value for each sub-volume.

Here, the sub-volume property value optimization unit 1700 may include a sub-volume selection module 1720 selecting at least one sub-volume to optimize the property value from respective volumes of which property values are determined; a sub-volume property value determination module 1740 optimizing a property value of the selected sub-volume to minimize the sum of squared differences between the measured values of measurement result derivation unit and the simulated values to determine the property value of the selected volume.

As described above, for each volume whose physical property values have been determined in the sub-volume selection module 1720, at least one sub-volume to optimize the physical property values may be selected, and the process for optimizing the physical property values may be repeatedly performed in order to minimize the sum of squared differences between the measured values of the measurement result derivation modules 1500 and the simulated values for the selected sub-volume such that the physical property values of the selected sub-volume by the sub-volume property value determination module 1740 is determined.

In particular, the sub-volume of the sub-volume property value optimization unit 1700 may be at least one voxel forming a cube. For example, one voxel may be defined as a sub-volume, or it may be defined as a sub-volume of a cube consisting of two, four, six, or eight voxels.

Here, the property value of each sub-volume may be at least one of electrical conductivity and permittivity

In addition, the minimizing of the sum of squared differences between the measured values of the measurement result derivation unit and the simulated values in the sub-volume property value optimization unit 1700 may be performed by running the simulation repeatedly while changing the property value assigned to the selected sub-volume to determine the property value of the selected sub-volume by using the nonlinear least-squares method.

Referring to FIGS. 2 through 4, a method for optimizing physical property values for each homogeneous property volume in a 3D subject according to an embodiment of the inventive concept, wherein the property values represent intrinsic properties of substances constituting the subject, may include a step of homogeneous property volume division S2100 dividing the 3D subject into volumes based on distinct homogeneous properties; a step of initial property value allocation S2200 assigning an initial property value of each of the divided volumes; a step of electrode arrangement S2300 placing a plurality of electrodes on the surface of the 3D subject; a step of measurement condition set S2400 setting at least one of voltage and current applied to the plurality of electrodes; a step of measurement result derivation S2500 applying at least one of voltage and current to the plurality of electrodes in accordance with the setting in the step of measurement condition set and measuring at least one of voltage and current generated on the plurality of electrodes; and a step of volume property value optimization S2600 performing a simulation based on the same conditions as the step of electrode arrangement S2300 and the step of measurement condition set S2400, and determining the property value for each volume to minimize the sum of squared differences between the measured values in the step of the measurement result derivation S2500 and the simulated values.

The step of homogeneous property volume division S2100 may be to divide the 3D subject into homogeneous material volumes with distinct material properties.

Here, the property value of each volume may be at least one of electrical conductivity and permittivity, and consequently, the division by the homogeneous property volume may be to divide the part with homogeneous electrical conductivity into one volume, or to divide the part with homogeneous permittivity into one volume.

The step of the initial property value allocation S2200 may assign the initial property for each of the divided volumes. In particular, the initial property value assigned for each of the divided volumes may be either the electrical property value measured by the conductivity measurement method or the electrical property value known about the tissue of the human body, in advance.

The step of electrode arrangement S2300 may place a plurality of electrodes on the surface of the 3D subject. A plurality of the electrodes may be arranged on the most efficient place in order to attach a plurality of electrodes on a body and repeat the injection of current and the measurement of voltage according to the electrical impedance imaging(EIT).

The step of measurement condition setting S2400 may set at least one of voltage and current applied to the plurality of the electrode. As the above step of the electrode arrangement S2300, at least one of voltage and current may be set in order to perform the most efficient measurement through the EIT.

The step of measurement result derivation S2500 may apply at least one of voltage and current to the plurality of electrodes according to the step of the measurement condition set S2400 and measure at least one of voltage and current generated on the plurality of electrodes.

In other words, the step of measurement result derivation S2500 may measure the current flowing to the plurality of electrode in the case of applying voltage to the plurality of electrodes, or measure the voltage between the plurality of electrodes in the case of applying current to the plurality of electrodes according to the step of measurement condition set S2400.

The step of volume property value optimization S2600 may perform a simulation under the same conditions as the step of electrode arrangement S2300, the step of measurement condition set S2400 and the step of measurement result derivation S2500, and determine the property value for each volume in order to minimize the sum of squared differences between the measured values in the step of measurement result derivation S2500 and the simulated values.

In addition, the step of volume property value optimization 2600 may include a volume selection process S2620 selecting at least one volume to optimize the property value among the plurality of the volumes; and a volume property value determination process S2640 optimizing a property value of the selected volume to minimize the sum of squared differences between the measured values in the measurement result derivation and the simulated values to determine the property value of the selected volume.

On the other hand, the method may further include a step of sub-volume property value optimization S2700 dividing the volume into a plurality of sub-volumes by using the property value of each volume determined in the step of volume property value optimization S2600, and optimizing property value for each sub-volume.

In addition, the step of sub-volume property value optimization S2700 may include a sub-volume selection process S2720 selecting at least one sub-volume to optimize the property value from respective volumes of which property value is determined: and a sub-volume property value determination process S2740 optimizing a property value of the selected sub-volume to minimize the sum of squared differences between the measured values in the step of measurement result derivation and the simulated values to determine the property value of the selected volume.

FIGS. 5A to 5C illustrate an embodiment of a method of dividing the volume into a plurality of sub-volumes. In FIG. 5A, the volume represented by σ1 may be divided into plurality of sub-volumes σ11, [symbol] , σij, [symbol] as shown in FIG. 5B or FIG. 5C, and the step of sub-volume property value optimization may be further implemented to optimize the property value for each sub-volume.

FIG. 6 is a schematic view illustrating an apparatus for optimizing property values for each homogeneous property volume in a 3D subject according to another embodiment of the inventive concept.

FIG. 7 shows a view of the model divided by tissue types in an apparatus for optimizing physical property values for each homogeneous property volume in a 3D subject according to another embodiment of the inventive concept. A dividing of the subject by tissue type S100 may divide the medical image by tissue types using a segmentation, and a 3D model may be created using the segmentation data. The sequence of inputting the initial property value S200 into the divided data may be a step of inputting the electrical property value known about the tissue as the initial property value into the generated 3D model. The known electrical property value may be referred to the literature, and other measuring instruments such as EIT (Electric Impedance Tomography), MR-EIT, DTI, DTI-EIT, etc. may be used.

Once the electrical property value for each tissue is entered, the number of electrodes may be determined according to the measurement method in the step of electrode arrangement S300. The measurement may be implemented by attaching two or more electrodes and may consist of an array of one or more electrodes. In a step S400 of calculating an error between the measured data and a calculated data, it may be determined whether the error is below a certain value and an optimization process may be implemented using the measured data. If the error value between the measured data and an initial electrical property data is large, the electrical property value of the tissue type may be reconstructed in the step of electrical property allocation S600 of the segmentation data. Through the reconstruction step, an optimized electrical property value for each tissue type may be finally obtained.

FIGS. 8A and 8B show a detailed view of the model divided by tissue types in an apparatus for optimizing physical property values for each homogeneous property volume in a 3D subject according to another embodiment of the inventive concept. In an embodiment, the tissue type was classified into 5 types, FIG. 8A shows the appearance and cross-section of the classified tissue types, and FIG. 8B shows the tissue type divided into an exterior cuboid 111, a cube 112, a cuboid 113, a sphere 114, and a cylinder 115 by volume and individual electrodes Electrode 1 through 8.

FIGS. 9A to 9C show an apparatus for optimizing the property value for each homogeneous property volume of a 3D subject according to another embodiment of the inventive concept, FIG. 9A shows the name of each side A1˜A4, and FIG. 9B shows the name of the individual electrode of each surface. FIG. 9C shows an electrode through which an electric current is applied to an object and an electrode to measure the voltage at that time. FIG. 9C is a table of current sources, grounds, and electrodes for measurement, where the Configuration means a direction in which the current is applied, the Current Path means the direction of the current flow, and in this embodiment, the A2 to A3(Ground) means that the electrode on the A2 side is applied with current, and A3 is the ground electrode. The Electrodes for Measurement means a measurement electrode for measuring the voltage between the ground and the measurement electrode when an electric current is applied.

FIGS. 10A and 10B show a data measurement and reconstruction according to the electrical properties through a 3D model in an apparatus for optimizing physical property values for each homogeneous property volume in a 3D subject according to another embodiment of the inventive concept. FIG. 10A is an error for optimizing the sum of squared differences between the measured data and the calculated data in the simulation, and the closer the error is to zero, the closer it is to the correct answer. In this embodiment, through the error optimization of 462 times, the electrical property value with the error value closest to 0 was obtained. FIG. 10B refers to an initial conductivity which randomly sets the conductivity for each tissue type, a final conductivity through the optimization, and a true conductivity which can be compared with the final conductivity.

FIG. 11 is a view illustrating a method for optimizing physical property values for each homogeneous property volume in a 3D subject according to further another embodiment of the inventive concept. In order to optimize the electrical property value, the data applied to the subject according to the position of the electrode may be measured in first S601, and a virtual 3D model including the electrical property of the measured subject may be created S602, and then applied data may be calculated according to the position of the electrode in the generated 3D model S603. After determining whether the error is within the permissible range S604 through calculating the difference between the actual measured data of the subject and the calculated data through the 3D model of the generated subject S603, if the error is not within the permissible range, after modifying the electrical property for each type of tissue S606, and then the applied data according to the position of the electrode of the generated 3D model is calculated S603 and repeat the flow chart to obtain the optimal electrical property value.

While the inventive concept has been described with reference to accompanying drawings, it will be apparent to those skilled in the art that various changes and modifications may be made without departing from the spirit and scope of the inventive concept. For example, a person skilled in the art may change the material, size, etc. of each component according to the field of application, or combine or replace the embodiments disclosed and perform them in a form that is not clearly disclosed in the embodiment of the present invention, however, this does not depart from the scope of the present invention. Thus, the scope of the inventive concept is to be determined by the broadest permissible interpretation of the following claims and their equivalents.
1100 Homogeneous property volume division unit
1200 Initial property value allocation unit
1300 Electrode arrangement unit
1400 Measurement condition setting unit
1500 Measurement result derivation unit
1600 Volume property value optimization unit
1620 Volume selection module
1640 Volume property value determination module
1700 Sub-volume property value optimization unit
1720 Sub-volume selection module
1740 Sub-volume property value determination module

1. An apparatus for optimizing physical property values for each homogeneous property volume in a 3D subject, wherein the property values represent intrinsic properties of substances constituting the subject, comprising:
a homogeneous property volume division unit dividing the 3D subject into volumes based on distinct homogeneous physical properties;
an initial property value allocation unit assigning an initial physical property value for each of the divided volumes;
an electrode arrangement unit placing a plurality of electrodes on a surface of the 3D subject;
a measurement condition setting unit setting at least one of voltage and current applied to the plurality of electrodes;
a measurement result derivation unit applying at least one of voltage and current to the plurality of electrodes in accordance with the setting of the measurement condition setting unit and measuring at least one of voltage and current generated on the plurality of electrodes; and
a volume property value optimization unit performing a simulation based on the same conditions as the electrode arrangement unit and the measurement condition setting unit, and determining the optimal property value for each volume by minimizing the differences between the measured values obtained by the measurement result derivation unit and the simulated values..

2. The apparatus for optimizing physical property values for each homogeneous property volume in a 3D subject of claim 1, wherein the volume property value optimization unit comprises:
a volume selection module selecting at least one volume to optimize the physical property value among the plurality of the volumes; and
a volume property value determination module optimizing a physical property value of the selected volume to minimize the differences between the measured values of the measurement result derivation unit and the simulated values to determine the physical property value of the selected volume.

3. The apparatus for optimizing physical property values for each homogeneous property volume in a 3D subject of claim 1, wherein the homogeneous property volume division unit receives a medical image of the 3D subject, extracts the outline of the subject based on the received medical image, and divides the 3D subject into volumes based on the distinct homogeneous physical properties.

4. The apparatus for optimizing physical property values for each homogeneous property volume in a 3D subject of claim 1, wherein the homogeneous property volume division unit receives medical image information divided by volumes based on the homogeneous physical properties.

5. The apparatus for optimizing physical property values for each homogeneous property volume in a 3D subject of claim 1, wherein current flowing to the plurality of electrodes is measured when voltage is applied to the plurality of electrodes based on the setting in the measurement result derivation unit.

6. The apparatus for optimizing physical property values for each homogeneous property volume in a 3D subject of claim 1, wherein, voltage between the plurality of electrodes is measured when current is applied to the plurality of electrodes based on the setting in the measurement result derivation unit.

7. The apparatus for optimizing physical property values for each homogeneous property volume in a 3D subject of claim 1, wherein, in the initial property value allocation unit, the initial physical property value assigned for each of the divided volumes is either the electrical property value measured by the conductivity measurement method or the electrical property value known about the tissue of the human body in advance.

8. The apparatus for optimizing physical property values for each homogeneous property volume in a 3D subject of claim 1, wherein the homogeneous property volume division unit segments the 3D subject into anatomical volumes based on distinct homogeneous properties using at least one of magnetic resonance imaging (MRI) or computed tomography (CT).

9. The apparatus for optimizing physical property values for each homogeneous property volume in a 3D subject of claim 8, wherein the anatomic volume includes at least one of skin, skull, white matter, gray matter of the mammalian brain, and multiple internal organs, bones, muscles and fat of the mammalian chest and abdomen.

10. The apparatus for optimizing physical property values for each homogeneous property volume in a 3D subject of claim 1, wherein the property value of each volume is at least one of electrical conductivity and permittivity.

11. The apparatus for optimizing physical property values for each homogeneous property volume in a 3D subject of claim 1, wherein, in the volume property value optimization unit, the minimizing of the sum of squared differences between the measured values of the measurement result derivation unit and the simulated values comprises performing the simulation repeatedly while changing the property value assigned to the selected volume, using the nonlinear least-squares method to determine the property value of the selected volume.

12. The apparatus for optimizing physical property values for each homogeneous property volume in a 3D subject of claim 1, further comprising:
a sub-volume property value optimization unit subdividing each homogeneous property volume into a plurality of sub-volumes, and optimizing physical property value for each sub-volume.

13. The apparatus for optimizing physical property values for each homogeneous property volume in a 3D subject of claim 12, wherein the sub-volume property value optimization unit comprises:
a sub-volume selection module selecting at least one sub-volume to optimize the property value from respective volumes of which property values are determined; and
a sub-volume property value determination module optimizing the property value of the selected sub-volume to minimize the sum of squared differences between the measured values of the measurement result derivation unit and the simulated values to determine the property value of the selected sub-volume.

14. The apparatus for optimizing physical property values for each homogeneous property volume in a 3D subject of claim 12, wherein, in the sub-volume property value optimization unit, the sub-volume is at least one voxel forming a cube.

15. The apparatus for optimizing physical property values for each homogeneous property volume in a 3D subject of claim 12, wherein the property value of each sub-volume is at least one of electrical conductivity and permittivity.

16. The apparatus for optimizing physical property values for each homogeneous property volume in a 3D subject of claim 12, wherein, in the sub-volume property value optimization unit, the minimizing of the sum of squared differences between the measured values of the measurement result derivation unit and the simulated values comprises performing the simulation repeatedly while changing the property value assigned to the selected sub-volume to determine the property value of the selected sub-volume by using the nonlinear least-squares method.

17. A method for optimizing physical property values of each homogeneous property volume in a 3D subject, wherein the physical property values represent intrinsic properties of substances constituting the subject, comprising:
a step of homogeneous property volume division dividing the 3D subject into volumes based on distinct homogeneous properties;
a step of initial property value allocation assigning an initial physical property value of each of the divided volumes;
a step of electrode arrangement placing a plurality of electrodes on the surface of the 3D subject;
a step of measurement condition setting at least one of voltage and current applied to the plurality of electrodes;
a step of measurement result derivation applying at least one of voltage and current to the plurality of electrodes in accordance with the setting in the step of measurement condition set and measuring at least one of voltage and current generated on the plurality of electrodes; and
a step of volume property value optimization performing a simulation based on the same conditions as the step of electrode arrangement and the step of measurement condition set, and determining the property value for each volume to minimize the differences between measured values in the measurement result derivation and the simulated values of the simulation.

18. The method for optimizing physical property values of each homogeneous property volume in a 3D subject of claim 17, wherein the step of volume property value optimization comprises:
a volume selection process selecting at least one volume to optimize the property value among the plurality of the volumes; and
a volume property value determination process optimizing a property value of the selected volume to minimize the differences between the measured values in the measurement result derivation and the simulated values to determine the property value of the selected volume.

19. The method for optimizing physical property values in a 3D subject of claim 17, further comprising:
a step of sub-volume property value optimization subdividing each homogeneous property volume, and optimizing property value for each sub-volume.

20. The method for optimizing physical property values of each homogeneous property volume in a 3D subject of claim 19, wherein the step of sub-volume property value optimization comprises:
a sub-volume selection process selecting at least one sub-volume to optimize the property value from respective volumes of which property values are determined; and
a sub-volume property value determination process optimizing a property value of the selected sub-volume to minimize the differences between the measured values in the step of measurement result derivation and the simulated values to determine the property value of the selected volume.
